SEQUENCE LISTING (16 sequences)

SEQ ID NO: 1 - RNA, 19 nt, Artificial Sequence
  Dual-targeted siRNA (antisense c-MET)
accacacaucugacuuggu

SEQ ID NO: 2 - RNA, 19 nt, Artificial Sequence
  Dual-targeted siRNA (antisense PD-L1)
accaauucagcuguauggu

SEQ ID NO: 3 - RNA, 50 nt, Artificial Sequence
  TTGGATCCAA loop shRNA
accacacaucugacuugguuuggauccaaaccaauucagcuguaugguuu

SEQ ID NO: 4 - RNA, 50 nt, Artificial Sequence
  TTCAAGAGAG loop shRNA
accacacaucugacuugguuucaagagagaccaauucagcuguaugguuu

SEQ ID NO: 5 - DNA, 50 bp, Artificial Sequence
  TTGGATCCAA loop shRNA-DNA insert
accacacatctgacttggtttggatccaaaccaattcagctgtatggttt

SEQ ID NO: 6 - DNA, 50 bp, Artificial Sequence
  TTCAAGAGAG loop shRNA-DNA insert
accacacatctgacttggtttcaagagagaccaattcagctgtatggttt

SEQ ID NO: 7 - DNA, 276 bp, Artificial Sequence
  U6 promoter
cctagagtcgacactagataacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaa

SEQ ID NO: 8 - DNA, 455 bp, Artificial Sequence
  hTERT promoter
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 9 - DNA, 900 bp, Artificial Sequence
  E1A
acaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtga

SEQ ID NO: 10 - DNA, 572 bp, Artificial Sequence
  IRES
cccctctccctcccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 11 - DNA, 1797 bp, Artificial Sequence
  E1B
catggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattga

SEQ ID NO: 12 - DNA, 3762 bp, Artificial Sequence
  hTERT-E1A-IRES-E1B
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgactagtacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgacccctctccctcccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattga

SEQ ID NO: 13 - DNA, 50 bp, Artificial Sequence; identical to SEQ ID NO: 5
  TTGGATCCAA loop shRNA coding sequence

SEQ ID NO: 14 - DNA, 50 bp, Artificial Sequence; identical to SEQ ID NO: 6
  TTCAAGAGAG loop shRNA coding sequence

SEQ ID NO: 15 - DNA, 10 bp, Artificial Sequence
  TTCAAGAGAG loof
ttcaagagag

SEQ ID NO: 16 - DNA, 10 bp, Artificial Sequence
  TTGGATCCAA loof
ttggatccaa

(19) United States
(12) Patent Application Publication
(10) Pub. No.: US 2024/0158789 A1
(43) Pub. Date: May 16, 2024

(54) NUCLEIC ACIDS SIMULTANEOUSLY INHIBITING EXPRESSION OF C-MET GENE AND PD-L1 GENE

(71) Applicant: CURIGIN CO., LTD., Seoul (KR)

(72) Inventors: Jin Woo CHOI, Seongnam-si (KR); Jung Ki YOO, Yangju-si (KR); Ki Hwan UM, Ansan-si (KR)

(73) Assignee: CURIGIN CO., LTD., Seoul (KR)

(21) Appl. No.: 18/281,539

(22) PCT Filed: Mar. 11, 2022
(86) PCT No.: PCT/KR2022/003454

§ 371 Date: Sep. 11, 2023

(30) Foreign Application Priority Data
Mar. 12, 2021 (KR) 10-2021-0032716

(51) Int. Cl. C12N 15/113; A61K 31/7105; A61K 45/06; A61P 35/00; C12N 15/86

(52) U.S. Cl. CPC C12N 15/113; A61K 31/7105; A61K 45/06; A61P 35/00; C12N 15/86; C12N 2310/122; C12N 2310/14; C12N 2710/10043

(57) ABSTRACT

The present invention relates to nucleic acid molecules simultaneously inhibiting the expression of a C-MET gene and a PD-L1 gene, and an anti-cancer pharmaceutical composition comprising same. All of the bispecific nucleic acid molecules of the present invention, designed so that the sense strand inhibits the expression of PD-L1 gene and the antisense strand inhibits the expression of c-MET gene, simultaneously inhibit the expression of a c-MET gene and a PD-L1 gene when actually applied as siRNA or shRNA to various cancer cells and remarkably inhibit the expression of both genes even when applied as shRNA to viruses, and thus are usable as an anti-cancer composition or an anti-cancer adjuvant for a variety of carcinomas, can be locally delivered, and having excellent selectivity.

### TECHNICAL FIELD

The present invention relates to nucleic acid molecules simultaneously inhibiting the expression of a C-MET gene and a PD-L1 gene, and an anti-cancer pharmaceutical composition comprising same.

### BACKGROUND ART

Cancer is one of diseases that cause the most deaths worldwide, and the development of innovative cancer therapy is able to reduce medical costs incurred during treatment of the cancer and create high added value at the same time. In addition, according to statistics from 2008, molecular therapeutic agents that may overcome resistance to existing anti-cancer drugs accounted for $17.5 billion in seven major countries (US, Japan, France, Germany, Italy, Spain, and UK), and in 2018, accounted for the market size of approximately $45 billion, which is expected to show a growth rate of 9.5% compared to 2008. Cancer treatment is divided into surgery, radiation therapy, chemotherapy, and biological therapy. Among them, the chemotherapy is therapy that inhibits or kills the proliferation of cancer cells using chemicals, and the toxicity caused by anti-cancer drugs also occurs in normal cells to exhibit a certain degree of toxicity, and even if anti-cancer drugs are effective, resistance develops because the effect is lost after a certain period of use. Therefore, there is an urgent need to develop anti-cancer drugs that act selectively on cancer cells and do not develop resistance (Current status of conquering cancer, Biowave 2004. 6(19)). Recently, new anti-cancer drugs targeting the molecular characteristics of cancer have been developed through the acquisition of molecular genetic information about cancer, and there are also reports that anti-cancer drugs targeting characteristic molecular targets only to cancer cells do not develop drug resistance.

The technology of inhibiting gene expression is an important tool in the development of therapeutic agents and validation of targets for disease treatment. Since its role was discovered, RNA interference (hereinafter referred to as ‘RNAi’) has been shown to act on sequence-specific mRNAs in various types of mammalian cells (Silence of the transcripts: RNA interference in medicine. J Mol Med (2005) 83: 764773). RNAi is a phenomenon in which small interfering RNA (hereinafter referred to as siRNA′) having a double helix structure of 21 to 25 nucleotides in size specifically binds to an mRNA transcript having a complementary sequence to decompose the corresponding transcript and inhibit the expression of the specific protein. In cells, RNA double strand is processed by an endonuclease called Dicer to be converted to siRNA of a double strand of 21 to 23 base pairs (bps), and siRNA binds to an RNA-induced silencing complex (RISC) so that a guide (antisense) strand sequence-specifically inhibits the expression of the target gene through the process of recognizing and decomposing the target mRNA (NUCLEIC-ACID THERAPEUTICS: BASIC PRINCIPLES AND RECENT APPLICATIONS. Nature Reviews Drug Discovery. 2002. 1, 503-514). According to a Bertrand's research team, it was found that siRNA for the same target gene had a superior inhibitory effect on mRNA expression in vitro and in vivo compared to antisense oligonucleotide (ASO) and included a long-lasting effect (Comparison of antisense oligonucleotides and siRNAs in cell culture and in vivo. Biochem. Biophys. Res. Commun. 2002. 296: 1000-1004). A RNAi technology-based therapeutics market including siRNA has been analyzed to form a total of over 12 trillion won of the later global market around 2020, and objects to which the corresponding technology may be applied have been dramatically expanded to be evaluated as a next-generation gene therapy technology capable of treating diseases that are hardly treated with existing antibody and compound-based medicines. In addition, since a mechanism of action of siRNA complementarily binds to the target mRNA to regulate the expression of the target gene in a sequence-specific manner, compared to a long development period and development cost required for existing antibody-based medicines or small molecule drugs to be optimized for a specific protein target, the applicable target may be dramatically expanded, and while the development period is shortened, there is an advantage of being able to develop lead compounds optimized for all protein targets, including non-medicatable target substances (Progress Towards in Vivo Use of siRNAs. MOLECULAR THERAPY. 2006 13(4):664-670). Therefore, recently, this RNA-mediated interference phenomenon presents a solution to the problem that occurs in the development of existing chemical synthetic medicines, and studies to selectively inhibit the expression of specific proteins at the transcript level to be used for the development of various disease therapeutic agents, especially tumor therapeutic agents are in progress. In addition, unlike conventional anti-cancer agents, siRNA therapeutics have an advantage of having a clear target and predictable side effects, but in the case of tumors, which are diseases caused by problems of various genes, such target specificity may rather cause a low therapeutic effect.

### DISCLOSURE

### Technical Problem

An object of the present invention is to provide bispecific nucleic acid molecules.

Another object of the present invention is to provide a recombinant expression vector including the nucleic acid molecules of the present invention and a virus introduced with the recombinant expression vector.

Yet another object of the present invention is to provide a pharmaceutical composition for preventing or treating cancer.

Yet another object of the present invention is to provide a use for preventing or treating cancer.

Yet another object of the present invention is to provide a method for treating cancer.

### Technical Solution

One aspect of the present invention provides a nucleic acid molecule simultaneously inhibiting a c-MET gene and a PD-L1 gene.

Another aspect of the present invention provides a recombinant expression vector including the nucleic acid molecule and a virus introduced with the recombinant expression vector.

Yet another aspect of the present invention provides a pharmaceutical composition for preventing or treating cancer including the nucleic acid molecule, a recombinant expression vector including the nucleic acid molecule, or a virus introduced with the recombinant expression vector as an active ingredient.

Yet another aspect of the present invention provides a use for preventing or treating cancer of the nucleic acid molecule of the present invention, a recombinant expression vector expressing the nucleic acid molecule, or a virus introduced with the vector.

Yet another aspect of the present invention provides a method for treating cancer of the nucleic acid molecule of the present invention, a recombinant expression vector expressing the nucleic acid molecule, or a virus introduced with the vector.

### Advantageous Effects

According to the present invention, all of the bispecific nucleic acid molecules, designed so that the sense strand inhibits the expression of PD-L1 gene and the antisense strand inhibits the expression of c-MET gene, simultaneously inhibit the expression of a c-MET gene and a PD-L1 gene when actually applied as siRNA or shRNA to various cancer cells and remarkably inhibit the expression of both genes even when applied as shRNA to viruses, and thus are usable as an anti-cancer composition or an anti-cancer adjuvant for a variety of carcinomas, can be locally delivered, and having excellent selectivity.

### DESCRIPTION OF DRAWINGS

FIG. 1 is a diagram illustrating a map of a vector for expressing shRNA including a bispecific siRNA set of the present invention in cells.

FIG. 2 is a diagram illustrating a vector map of an adenovirus vector CA104 of the present invention.

FIG. 3 is a diagram confirming an effect of inhibiting the expression of c-MET and PD-L1 genes by a bispecific siRNA (double strand) set capable of simultaneously inhibiting c-MET and PD-L1 of the present invention in various cancer cell lines.

FIG. 4 is a diagram of confirming the effect of inhibiting the expression of c-MET and PD-L1 genes of the bispecific shRNA of the present invention.

FIG. 5 is a diagram of confirming the effect of inhibiting the expression of c-MET and PD-L1 genes by a recombinant adenovirus CA104 of the present invention including an hTERT promoter and a bispecific shRNA expression cassette.

FIG. 6 is a diagram of confirming a cancer cell killing effect of adenovirus expressing the bispecific shRNA of the present invention.

### BEST MODE FOR THE INVENTION

Hereinafter, exemplary embodiments of the present invention will be described in detail. However, the following exemplary embodiments are presented as examples of the present invention, and the present invention is not limited thereby, and various modifications and applications of the present invention can be made within the description of claims to be described below and equivalents interpreted therefrom.

Unless otherwise indicated, a nucleic acid is recorded in a 5′—>3′ direction from left to right. A numerical range enumerated within the specification is inclusive of numbers defining the range and includes each integer or any non-integer fraction within a defined range.

Unless defined otherwise, all technical and scientific terms used herein have the same meaning as commonly understood by those skilled in the art to which the present invention pertains. Although any methods and materials similar or equivalent to those described herein can be used in the practice for testing the present invention, the preferred materials and methods are described herein.

In one aspect, the present invention relates to nucleic acid molecules simultaneously inhibiting the expression of a Programmed death-ligand 1 (PD-L1) gene and a Homo sapiens MET proto-oncogene (c-MET) gene.

In one embodiment, a nucleotide sequence inhibiting the c-MET gene and a nucleotide sequence inhibiting PD-L1 may partially form a complementary bond.

In one embodiment, a nucleotide sequence having 80% or more homology to a nucleotide sequence represented by SEQ ID NO: 1 or 2 may be included.

In one embodiment, a nucleic acid molecule including the nucleotide sequence represented by SEQ ID NO: 1 may inhibit the expression of the c-MET gene by RNA interference, and a nucleic acid molecule including the nucleotide sequence represented by SEQ ID NO: 2 may inhibit the expression of the PD-L1 gene by RNA interference, so that the nucleic acid molecules of the present invention may simultaneously inhibit the expression of the c-MET gene and the PD-L1 gene.

In one embodiment, the nucleic acid molecule may be double-stranded siRNA in which small interfering RNA (siRNA) containing the nucleotide sequence represented by SEQ ID NO: 1 and siRNA containing the nucleotide sequence represented by SEQ ID NO: 2 partially form a complementary bond. In one example of the present invention, siRNA (antisense strand for the c-MET gene) consisting of the nucleotide sequence represented by SEQ ID NO: 1 and siRNA (antisense strand for the PD-L1 gene) consisting of the nucleotide sequence represented by SEQ ID NO: 2 were fabricated so that 15 mer of 19 mer is complementary bound to form double-stranded siRNA. It was confirmed that the double-stranded siRNA targets the c-MET gene and the PD-L1 gene, respectively, to simultaneously inhibit the expression of both the genes, which is a bispecific siRNA set.

In one embodiment, the nucleic acid molecules may be short hairpin RNA (shRNA) containing the nucleotide sequence represented by SEQ ID NO: 1 and the nucleotide sequence represented by SEQ ID NO: 2, and the shRNA may include a nucleotide sequence having at least 80% homology to a nucleotide sequence represented by SEQ ID NO: 3 or 4.

In one embodiment, the shRNA may have a hairpin structure in which the nucleotide sequence represented by SEQ ID NO: 1 and the nucleotide sequence represented by SEQ ID NO: 2 partially form a complementary bond to be linked palindromically by a loop region, and was described as TTCAAGAGAG loop shRNA (SEQ ID NO: 3) or TTGGATCCAA loop shRNA (SEQ ID NO: 4) according to a nucleotide sequence (SEQ ID NO: 15 or SEQ ID NO: 16) of the loop region of the hairpin structure in Example of the present invention.

In the present invention, siRNA targeting c-MET and PD-L1 has a sequence complementary to a part of the c-MET gene or PD-L1 gene of human (Homo sapiens), and may degrade mRNA of the c-MET gene or PD-L1 gene or inhibit translation.

As used in the present invention, the term “expression inhibition” means causing a decrease in expression (of mRNA) or translation (to protein) of a target gene, and preferably means that the expression of the target gene becomes undetectable or exists at an insignificant level thereby.

As used in the present invention, the term “small interfering RNA (siRNA)” refers to short double-stranded RNA capable of inducing a RNA interference (RNAi) phenomenon through cleavage of specific mRNA. Generally, siRNA consists of a sense RNA strand with a sequence homologous to mRNA of the target gene and an antisense RNA strand with a complementary sequence thereto. However, the double-stranded siRNA of the present invention has siRNA (antisense strand for the c-MET gene) in which a sense RNA strand consists of the nucleotide sequence represented by SEQ ID NO: 1, and siRNA (antisense strand for the PD-L1 gene) in which an antisense RNA strand consists of the nucleotide sequence represented by SEQ ID NO: 2. Accordingly, since the double-stranded siRNA may simultaneously inhibit the expression of the c-MET gene and the PD-L1 gene, the double-stranded siRNA is provided as an efficient gene knock-down method or a method of gene therapy.

As used in the present invention, the term “short hairpin RNA (shRNA)” refers to RNA in which a palindromic nucleotide sequence is partially included in single-stranded RNA to form a hairpin-like structure with a double-stranded structure in a 3′ region and expressed in the cell and then cleaved by dicer, a type of RNase present within the cell, and converted into siRNA. The length of the double-stranded structure is not particularly limited, but is preferably 10 nucleotides or more, and more preferably 20 nucleotides or more. In the present invention, the shRNA may be included in an expression cassette, and the shRNA may be produced by fabricating an expression cassette that encodes shRNA by converting U to T in a set sequence consisting of siRNA antisense strand and sense strand for each gene and then sequentially linking TTGGATCCAA (TTGGATCCAA loop) or TTCAAGAGAG (TTCAAGAGAG loop), an antisense strand and TT to 3′ of the sense strand, and expressing the expression cassette in the cell. In this case, the expression cassette may include a nucleic acid in which U is converted to T in the nucleotide sequence represented by SEQ ID NO: 1 and 2. Here, 15mers of a 19mer siRNA set consisting of SEQ ID NOs: 1 and 2 are complementary to each other. The siRNA of SEQ ID NO: 1 (antisense c-MET) binds complementary to mRNA of c-MET, and the siRNA of SEQ ID NO: 2 (antisense PD-L1) may bind complementary to mRNA of PD-L1.

In one embodiment, the expression cassette including the shRNA of the present invention may include a nucleotide sequence obtained by converting a RNA sequence represented by SEQ ID NO: 3 or 4 into a DNA sequence, and the DNA sequence may include SEQ ID NO: 5 or 6.

In one embodiment, the nucleotide sequence targeting PD-L1 may include a nucleotide sequence having at least 60% complementarity with a reverse complementary sequence of the nucleotide sequence targeting c-MET, and the nucleotide sequence targeting c-MET may include a nucleotide sequence having at least 60% complementarity with a reverse complementary sequence of the nucleotide sequence targeting PD-L1.

In one embodiment, the nucleotide sequence targeting PD-L1 may include a nucleotide sequence with complementarity of 70%, 80%, 85%, 90%, 95%, 96%, 97%, 98% or 99% or more with the reverse complementary sequence of the nucleotide sequence targeting c-MET, and the nucleotide sequence targeting c-MET may include a nucleotide sequence with complementarity of 70%, 80%, 85%, 90%, 95%, 96%, 97%, 98% or 99% or more with the reverse complementary sequence of the nucleotide sequence targeting PD-L1.

Variants of the nucleotide sequence targeting PD-L1 or the nucleotide sequence targeting c-MET are included within the scope of the present invention. The expression cassette of the present invention is a concept that includes variants capable of having the same functional action as the nucleotide sequence molecule, even though a functional equivalent of the nucleic acid molecules constituting the expression cassette, for example, some nucleotide sequences of the nucleic acid molecules have been modified by deletion, substitution, or insertion. The “% of sequence homology” with the nucleic acid molecules is determined by comparing two optimally arranged sequences with a comparison region, and a part of the nucleic acid molecule sequence in the comparison region may include addition or deletion (i.e., gap) compared to a reference sequence (not including addition or deletion) for an optimal alignment of the two sequences.

In one aspect, the present invention relates to a recombinant expression vector expressing the nucleic acid molecules of the present invention.

In one embodiment, the recombinant expression vector of the present invention may include a nucleotide sequence encoding siRNA containing the nucleotide sequence represented by SEQ ID NO: 1 and siRNA containing the nucleotide sequence represented by SEQ ID NO: 2, and include a nucleotide sequence (DNA sequence) encoding shRNA containing the nucleotide sequence represented by SEQ ID NO: 3 or shRNA containing the nucleotide sequence represented by SEQ ID NO: 4.

The recombinant vector of the present invention may be produced by recombinant DNA methods known in the art, and in one example, a pE3.1 vector was used.

In the present invention, non-viral vectors useful for delivering siRNA for c-MET and PD-L1 include all vectors commonly used in gene therapy, and include, for example, various plasmids and liposomes that may be expressed in eukaryotic cells.

In the present invention, in order to allow double-stranded siRNA targeting c-MET and PD-L1 to be properly transcribed in the delivered cells, it is preferred that the nucleotide sequence encoding shRNA containing c-MET and PD-L1, particularly, shRNA consisting of the nucleotide sequence represented by SEQ ID NO: 3 or the nucleotide sequence represented by SEQ ID NO: 4 is at least operably linked to a promoter. The promoter may be any promoter that is functionable in eukaryotic cells, but more preferably a U6 promoter represented by SEQ ID NO: 7. For efficient transcription of double-stranded siRNA or shRNA targeting c-MET and PD-L1, if necessary, the promoter may also additionally include regulatory sequences including a leader sequence, a polyadenylation sequence, a promoter, an enhancer, an upstream activation sequence, a signal peptide sequence, and a transcription terminator.

In the present invention, viruses or viral vectors useful for delivering siRNA or shRNA for c-MET and PD-L1 include baculoviridiae, parvoviridiae, picomoviridiae, herepesviridiae, poxviridiae, adenoviridiae, and the like, but are not limited thereto.

In one aspect, the present invention relates to a virus introduced with the recombinant expression vector of the present invention.

In one embodiment, the virus of the present invention may be an anti-tumor adenovirus including a human telomere promoter (hTERT); and a recombinant expression vector including a nucleotide sequence targeting PD-L1 and a nucleotide sequence targeting c-MET.

In one embodiment, the virus of the present invention is an adenovirus, and the adenovirus is adenovirus including E1A and E1B operably linked to the hTERT promoter; and a nucleotide sequence encoding SEQ ID NO: 1 and SEQ ID NO: 2 in a E3 region, and when the nucleotide sequence is expressed, SEQ ID NO: 1 and SEQ ID NO: 2 may partially form a complementary bond to have a hairpin structure.

In one embodiment, the human telomere promoter may be operably linked to an endogenous gene of the adenovirus.

In the present invention, the term “operably linked” means a functional linkage between a gene expression regulatory sequence (e.g., a promoter, a signal sequence, or an array of transcriptional regulatory factor binding sites) and another gene sequence, and the regulatory sequence thereby regulates the transcription and/or translation of the another gene sequence.

In one embodiment, the hTERT promoter may include a nucleotide sequence represented by SEQ ID NO: 8.

In one embodiment, the endogenous gene of the adenovirus has a structure of 5′ITR-C1-C2-C3-C4-C5 3′ITR; in which the C1 includes E1A (SEQ ID NO: 9), E1B (SEQ ID NO: 11) or E1A-E1; the C2 includes E2B-L1-L2-L3-E2A-L4; the C3 does not include E3 or includes E3; the C4 includes L5; and the C5 may not include E4 or include E4, and may include a nucleotide sequence represented by SEQ ID NO: 12.

In one embodiment, the expression cassette may be located in a C3 region of the endogenous gene of the adenovirus.

In one embodiment, the hTERT promoter may be operably linked to E1A and E1B of the endogenous gene of the adenovirus.

In one embodiment, an IRES sequence (SEQ ID NO: 10) may be further included between E1A and E1B of the endogenous gene of the adenovirus.

In one aspect, the present invention relates to a pharmaceutical composition for preventing or treating cancer containing the nucleic acid molecules of the present invention, the recombinant expression vector of the present invention, or the virus of the present invention as an active ingredient.

In one embodiment, the composition may further include an anti-cancer agent.

In one embodiment, the anti-cancer agent may be at least one selected from the group consisting of dacomitinib, osimertinib, cetuximab, Pyrotinib, Lcotinib, panitumumab, zalutumumab, Nimotuzumab, matuzumab, gefitinib, erlotinib, Lapatinib, neratinib, vandetanib, necitumumab, afatinib, Taxol, Cisplatin, doxorubicin, paclitaxel, vincristine, topotecan, docetaxel, 5-fluorouracil (5-FU), gleevec, carboplatin, daunorubicin, valrubicin, flutamide, gemcitabine, and Etoposide.

In one embodiment, the cancer may be at least one selected from the group consisting of colorectal cancer, breast cancer, uterine cancer, cervical cancer, ovarian cancer, prostate cancer, brain tumor, head and neck carcinoma, melanoma, myeloma, leukemia, lymphoma, stomach cancer, lung cancer, pancreatic cancer, non-small cell lung cancer, liver cancer, esophageal cancer, small intestine cancer, perianal cancer, fallopian tube carcinoma, endometrial carcinoma, vaginal carcinoma, vulvar carcinoma, Hodgkin's disease, bladder cancer, kidney cancer, ureteral cancer, renal cell carcinoma, renal pelvic carcinoma, bone cancer, skin cancer, head cancer, cervical cancer, skin melanoma, intraocular melanoma, endocrine cancer, thyroid cancer, parathyroid cancer, adrenal cancer, soft tissue sarcoma, urethral cancer, penile cancer, central nervous system (CNS) tumor, primary CNS lymphoma, spinal cord tumor, brainstem glioma, and pituitary adenoma, more preferably glioblastoma, prostate cancer, melanoma, and non-small cell lung cancer.

As used in the present invention, the term “treatment” refers to any action that improves or beneficially changes the death of cancer cells or the symptoms of cancer by administering the composition containing the nucleic acid of the present invention. Those skilled in the art to which the present invention pertains will be able to determine the degree of improvement, enhancement and treatment by knowing the exact criteria of a disease for which the composition of the present invention is effective by referring to data presented by the Korean Academy of Medical Sciences, etc.

In one embodiment, the pharmaceutical composition may be one or more formulations selected from the group including oral formulations, external formulations, suppositories, sterile injection solutions and sprays.

The therapeutically effective dose of the composition of the present invention may vary depending on many factors, for example, an administration method, a target site, a condition of a patient, and the like. Accordingly, when used in the human body, the dose should be determined as an appropriate amount in consideration of both safety and efficiency. It is also possible to estimate the amount used in humans from the effective dose determined through animal experiments. These matters to be considered when determining the effective dose are described in, for example, Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

The composition of the present invention may include a carrier, a diluent, an excipient, or a combination of two or more thereof, which are commonly used in biological agents. The pharmaceutically acceptable carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these components, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be prepared preferably according to each disease or ingredient using as a suitable method in the art or a method disclosed in Remington's Pharmaceutical Science (Mack Publishing Company, Easton PA, 18th, 1990).

The composition of the present invention may further contain one or more active ingredients exhibiting the same or similar function. The composition of the present invention includes 0.0001 to 10 wt %, preferably 0.001 to 1 wt % of the protein, based on the total weight of the composition.

The pharmaceutical composition of the present invention may further include a pharmaceutically acceptable additive. At this time, the pharmaceutically acceptable additive may use starch, gelatinized starch, microcrystalline cellulose, lactose, povidone, colloidal silicon dioxide, calcium hydrogen phosphate, lactose, mannitol, syrup, gum arabic, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, sucrose, dextrose, sorbitol, talc and the like. The pharmaceutically acceptable additive according to the present invention is preferably included in an amount of 0.1 part by weight to 90 parts by weight based on the composition, but is not limited thereto.

The composition of the present invention may be administered parenterally (e.g., intravenously, subcutaneously, intraperitoneally or topically) or orally according to a desired method, and the dose may vary depending on the weight, age, sex, and health condition of a patient, a diet, an administration time, an administration method, an excretion rate, and the severity of a disease. A daily dose of the composition according to the present invention is 0.0001 to 10 mg/ml, preferably 0.0001 to 5 mg/ml, and more preferably administered once to several times a day.

Liquid formulations for oral administration of the composition of the present invention correspond to suspensions, internal solutions, emulsions, syrups, etc., and may include various excipients, such as wetting agents, sweeteners, fragrances, preservatives, and the like in addition to water and liquid paraffin, which are commonly used simple diluents. Formulations for parenteral administration include sterilized aqueous solutions, non-aqueous solvents, suspensions, emulsions, lyophilized agents, suppositories, and the like.

The pharmaceutical composition of the present invention may be used to prevent or treat cancer and its complications, and may also be used as an anti-cancer adjuvant.

In one aspect, the present invention provides a use for preventing or treating cancer of the nucleic acid molecules of the present invention, a recombinant expression vector expressing the nucleic acid molecules, or a virus introduced with the vector.

In one aspect, the present invention provides a method for treating cancer using the nucleic acid molecules of the present invention, a recombinant expression vector expressing the nucleic acid molecules, or a virus introduced with the vector.

### MODES FOR THE INVENTION

Hereinafter, the present invention will be described in more detail with reference to the following Examples. However, the following Examples are only intended to embody the contents of the present invention, and the present invention is not limited thereto.

### Example 1. Preparation of c-MET and PD-L1 Bispecific siRNA

A bispecific siRNA (double strand) set capable of simultaneously inhibiting c-MET (Homo sapiens MET proto-oncogene) and PD-L1 (Programmed death-ligand 1) was prepared with sequences shown in Table 1 below (Bioneer, Daejeon, Korea). Specifically, in a 19mer siRNA set 1 consisting of SEQ ID NOs: 1 and 2, 15mers were complementary to each other, and the siRNA of SEQ ID NO: 1 (antisense c-MET) in Table 1 below was bound complementary to mRNA of c-MET, and the siRNA of SEQ ID NO: 2 (antisense PD-L1) was bound complementary to mRNA of PD-L1. Therefore, the siRNA set of the present invention simultaneously reduced the expression of c-MET and PD-L1 genes.

TABLE 1

| | | | | | | | Comple- |
|---|---|---|---|---|---|---|---|
| | | | | | | | mentary |
| | Sequence | SEQ | | Sequence | SEQ | | bond |
| | (sense) | ID | | (antisense) | ID | length | length |
| siRNA | 5′-3′ | NO: | siRNA | 5′-3′ | NO: | (mer) | (mer) |
| c-MET | ACCACACAUC | 1 | PD-L1 | ACCAAUUCAGC | 2 | 19 | 15 |
| | UGACUUGGU | | | UGUAUGGU | | | |

### Example 2. Preparation of c-MET and PD-L1 Bispecific shRNA Expression Cassette

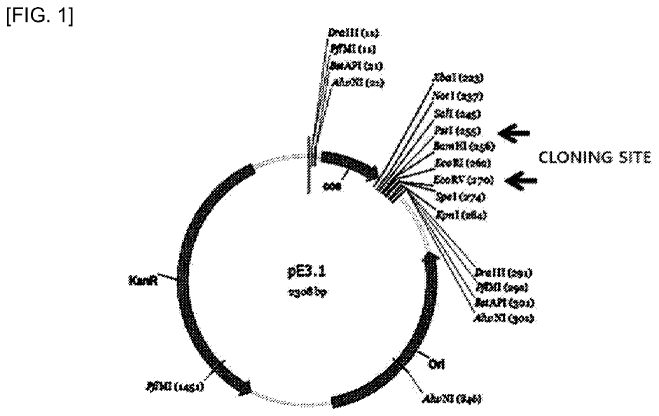

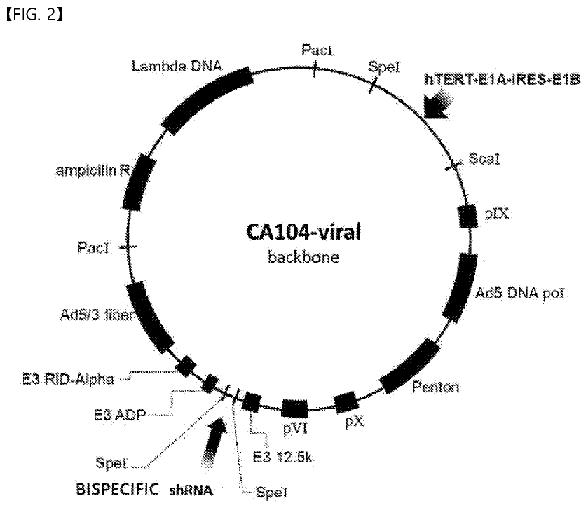

In order to be able to express the siRNA prepared in Example in cells, an shRNA expression cassette (TTGGATCCAA loop shRNA and TTCAAGAGAG loop shRNA) including a bispecific siRNA (SEQ ID NO: 1 and 2) double-stranded sequence and a loop sequence was prepared. Specifically, a DNA sequence encoding shRNA was prepared by linking TTGGATCCAA (TTGGATCCAA loop) or TTCAAGAGAG (TTCAAGAGAG loop), an antisense strand, and TT to 3′ of the sense strand of the siRNA set (SEQ ID NO: 1 and 2) in Table 1 above in s 5′ to 3′ direction, and indicated in Table 2 (siRNAs were indicated in uppercase letters and additional sequences were indicated in lowercase letters). The prepared shRNA expression cassettes were placed at cleavage sites of restriction enzymes PstI and EcoRV of the pE3.1 vector (FIG. 1), respectively, after the U6 promoter (SEQ ID NO: 7) to prepare a recombinant expression vector (shPDL1 &c-MET) that expressed two types of shRNA including bispecific siRNA targeting c-MET and PD-L1 in cells.

TABLE 2

| | | SEQ |
|---|---|---|
| | Sequence (5′→3′) | ID NO: |
| antisense c-MET | ACCACACAUCUGACUUGGU | 1 |
| antisense PD-L1 | ACCAAUUCAGCUGUAUGGU | 2 |
| TTGGATCCAA loop | ACCACACAUCUGACUUGGU | 3 |
| shRNA | uuggauccaaACCAAUUCA | |
| | GCUGUAUGGUuu | |
| TTCAAGAGAG loop | ACCACACAUCUGACUUGGU | 4 |
| shRNA | uucaagagagACCAAUUCA | |
| | GCUGUAUGGUuu | |

### Example 3. Preparation of Bispecific shRNA Coding Adenovirus

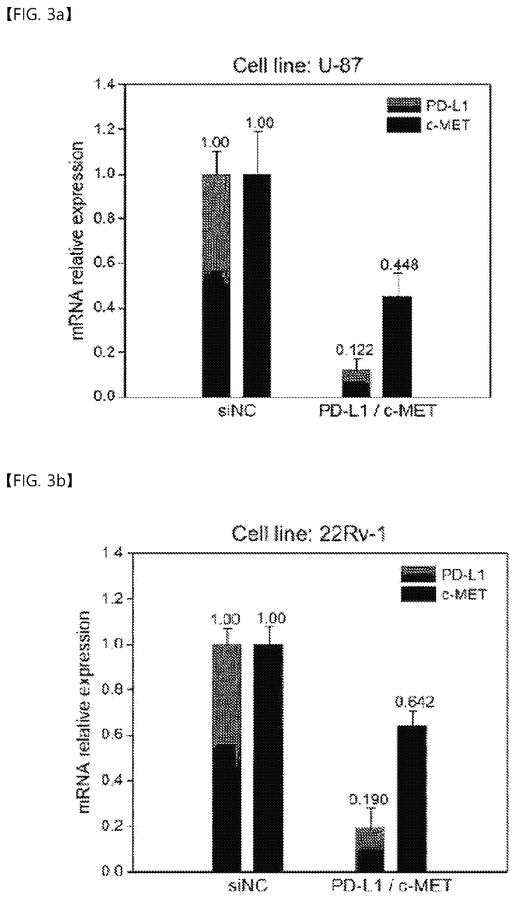

An infectious recombinant adenovirus was prepared by inserting a hTERT promoter (SEQ ID NO: 8)-E1A (SEQ ID NO: 9)-IRES (SEQ ID NO: 10)-E1B sequence (SEQ ID NO: 11) (full sequence: SEQ ID NO: 12) between SpeI and ScaI of the adenovirus vector, and then inserting the U6 promoter and a c-MET and PD-L1 bispecific shRNA coding sequence (SEQ ID NO: 13 or 14) prepared in Example above between SpeI and SpeI in each E3 region to express the hTERT promoter and the bispecific shRNA (c-MET and PD-L1 bispecific shRNA coding (expressing) adenovirus: CA104) (see FIG. 2). In addition, as a control group, a recombinant adenovirus inserted with only hTERT was also prepared (CA10G). Thereafter, the sequence of the prepared adenovirus vector was analyzed, and then if there were no abnormalities, a viral genome was linearized using a Pad restriction enzyme, and each virus was produced by transduction into 293A cells using a CaCl₂ method.

### Example 4. Confirmation of Gene Expression Inhibition Effect of Bispecific siRNA

A glioblastoma cell line U-87, a prostate cancer cell line CWR22Rv-1 (22Rv-1), a melanoma cell line A431, and a non-small cell lung cancer cell line HCC827 were each dispensed into a 12-well plate, and then incubated in a RPMI medium (Hyclone Co., Ltd.) added with 10% FBS (Hyclone Co., Ltd.) under 37° C. and 5% CO₂ conditions until the cell density reached 50%. Thereafter, the bispecific siRNA set prepared in Example 1 was transfected into the wells in which the cells were incubated with 3 μl of lipofectamine 3000 (Invitrogen, Carlsbad, CA, USA) at 80 pmole per well to simultaneously knock down c-MET and PD-L1. After 48 hours of transfection, each cell was lyzed and total RNA was extracted using a GeneJET RNA Purification Kit (Invitrogen). The extracted total RNA was used as a template to be reversely transcribed into cDNA through RT-PCR reaction, and then mRNA expression levels of c-MET and PD-L1 were confirmed using each siRNA and the bispecific siRNA set of the present invention through q-PCR reaction. To confirm the mRNA expression level, a primer set for PD-L1 or c-MET and a reaction mixture [10× reaction buffer 2 μl, HQ Buffer 2 μl, dNTP 1.6 μl, each primer (F, R, 10 pmole/μl) 1 μl, template (500 ng) 2 μl, Taq 0.2 μl, DW 10.2 μl, Total vol. 20 μl] were used. The mRNAs of c-MET and PD-L1 were converted into cDNA in a cell lysate knocked down under PCR conditions [2 min at 95° C., 30 cycles of 20 seconds at 95° C., 10 seconds at 60° C. and 30 to 60 seconds at 72° C., 5 minutes at 72° C.]. In addition, a reaction mixture [Template (RT-PCR product) 6 μl, Taqman probe 3 μl, 10× reaction Buffer 6 μl, HQ Buffer 6 μl, dNTP 4.84 Taq 0.6 μl, DW 10.2 μl, Total vol. 60 μl] was prepared using the reverse-transcribed cDNA as a template and qPCR was performed using QS3 equipment [10 minutes at 95° C., 15 seconds at 95° C. and 40 cycles per minute at 60° C.]. All reactions were repeated three times and average values thereof were taken. The results obtained above were normalized to mRNA values of a housekeeping gene GAPDH.

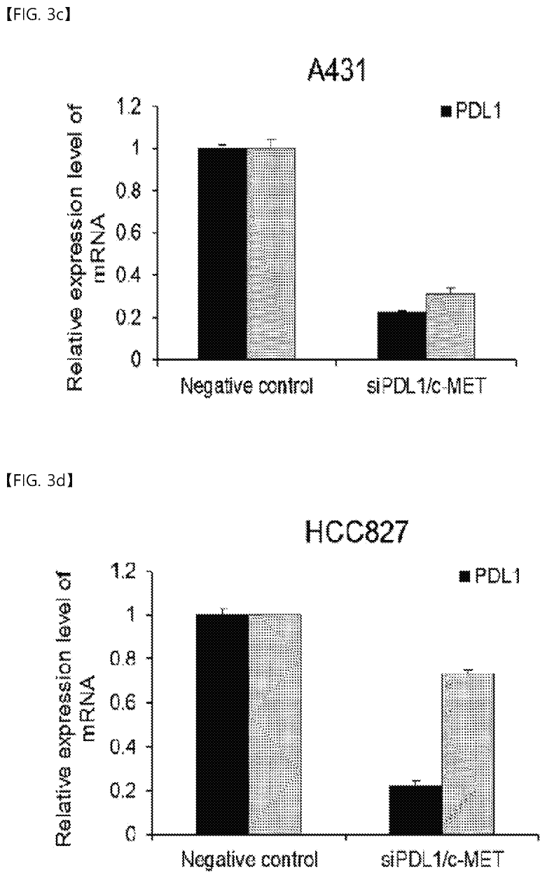

As a result, it was shown that the expression of c-MET and PD-L1 was reduced by the bispecific siRNA set of the present invention in all of the U-87 cell line, the 22Rv-1 cell line, the A431 cell line, and the HCC827 cell line (FIGS. 3A to 3D). Through this, it was found that the bispecific siRNA of the present invention may effectively inhibit the expression of the two genes at the same time.

### Example 5. Confirmation of Gene Expression Inhibition of Bispecific siRNA

The effect of inhibiting the expression of the target genes c-MET and PD-L1 of the bispecific shRNA prepared in Example 2 was confirmed. Specifically, a HeLa cell line (2×10⁵/well) was dispensed into a 6-well plate and then transfected into 2 μg of a negative control group plasmid (pAd1129-shNC) and an experimental group plasmid CA104-shRNA (shPDL1&c-MET) with Lipofectamine 3000 (Invitrogen) and incubated for 72 hours. After 3 days, RNA was extracted from the cells using Trizol (Thermo), quantified using Nanodrip, and the purity was confirmed. 400 ng of the RNA and DW were added to an RT premix (Intron) tube to make a mixture of 20 μl, and then cDNA was synthesized using PCR equipment at 45° C. for 1 hour and 95° C. for 5 minutes. 2 μl of the synthesized cDNA, 5 pmole of a bidirectional primer in Table 3, 10 μl of 2×SYBR green master mix (Bioline), and DW were mixed to make a mixture of 20 μl, and a PCR reaction was performed. The reaction conditions were performed 40 cycles at 50° C. for 2 minutes, 95° C. for 2 minutes, 95° C. for 5 seconds, and 60° C. for 30 seconds.

TABLE 3

| Gene name | Direction | Sequence (5′→3′) |
|---|---|---|
| POL1 | Forward | TCCAAGAGAGAGGAGAAGCTTTTC |
| | Reverse | CAGGATCTAATCTCCTAAAAGTGCAGTA |
| c-MET | Forward | TCCTCTGGGAGCTGATGACAA |
| | Reverse | TCCCTTGCAACAAGTAAACAGTTATATC |
| GAPDH | Forward | ATGCTGGCGCTGAGTACGT |
| | Reverse | AGCCCCAGCCTTCTCCAT |

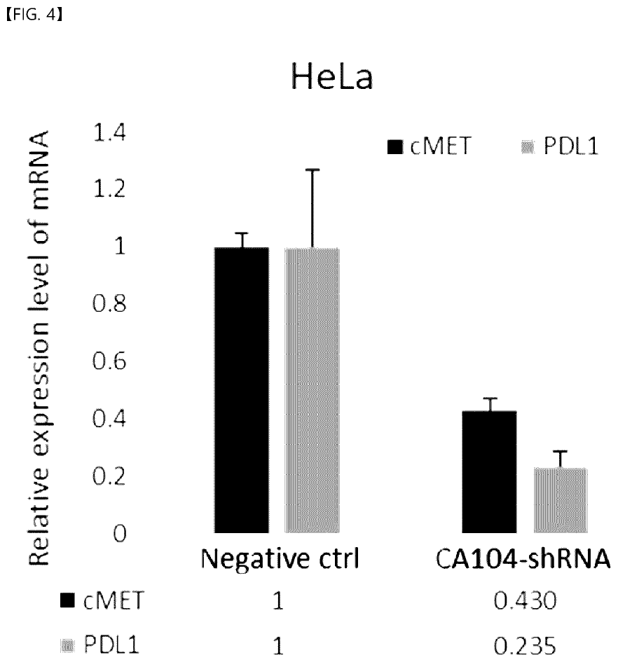

As a result, it was found that the expression of PD-L1 and cMET was significantly reduced in the cells transfected with the bispecific shRNA (shPDL1 &c-MET) of the present invention (FIG. 4).

### Example 6. Confirmation of Gene Expression Inhibition of Bispecific siRNA Coding Adenovirus

The effect of inhibiting the expression of the target genes c-MET and PD-L1 of the recombinant adenovirus CA104 prepared in Example 3 was confirmed. Specifically, a A431 cell line (1×10⁵/well) was dispensed into a 12-well plate, and then after 1 hour, treated with CA10G and CA104 in each well to be 2 to 5 MOI, and after 72 hours, RNA prep was performed using an RNA prep kit (Takara, 9767A). Thereafter, RNA was quantified using Nanodirp, added with 400 ng/20 μl per tube using an RT Premix (intron, 25081), and mixed well with a premix content, and then reacted at 45° C. for 1 hour and at 95° C. for 5 minutes using a PCR device to synthesize cDNA. A PCR mixture (total volume, 20 μl) corresponding to an experimental group was prepared using 2 μl of synthesized cDNA as a template (2 μl of template, 0.5 μl (10 pmole/μl) of forward primer, 0.5 μl (10 pmole/μl) of reverse primer, 10 μl of 2× master mix (Bioline, BIO-94005) and 7 μl of DW). The prepared PCR mixture was vortexed, mixed well, centrifuged, and then reacted in a qPCR device (Applied Biosystems, QS3) in 40 cycles for 5 minutes at 95° C., 10 seconds at 95° C., and 30 seconds at 60° C. The results were analyzed using a program on the qPCR device.

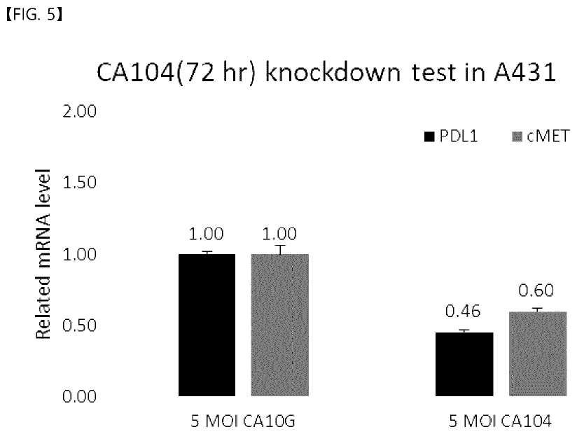

As a result, it was found that in the A431 cells, the recombinant adenovirus CA104 of the present invention encoding and expressing the hTERT promoter and the c-MET and PD-L1 bispecific shRNA significantly inhibited the expression of the c-MET and PD-L1 genes compared to the recombinant adenovirus CA10G including only the hTERT promoter (FIG. 5).

### Example 7. Confirmation of Cancer Cell Killing Effect of Bispecific siRNA Coding Adenovirus

A cancer cell killing effect of the recombinant adenovirus CA104 prepared in Example 3 was confirmed. Specifically, a Hela cell line was dispensed in a 96-well plate at 5×10 3 cells/well, and then infected by treating CA10G and CA104 at 1, 2, 5, 10, 20, 50, 100, and 200 MOI, respectively. After 72 hours of infection, the cells were photographed using a fluorescence microscope, and treated with 50 μl of an XTT solution (Roche) per well and incubated at 37° C. After 2 hours, the cell viability (%) of a virus-treated group compared to 0 MOI was calculated using an OD value obtained by measuring the absorbance at 450 nm wavelength and converted into a graph.

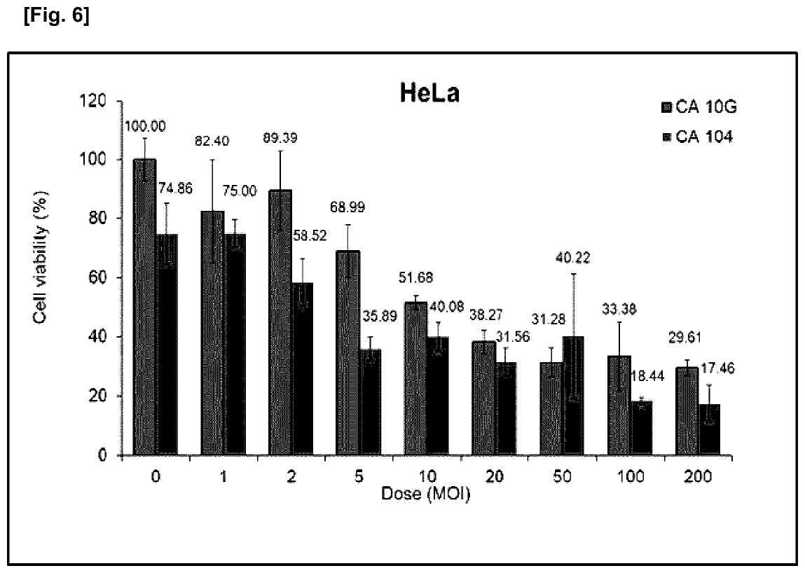

As a result, it was found that the cancer cell killing effect of CA104 was significantly greater than that of CA10G in the 2 MOI treated group and the 5 MOI treated group (FIG. 6).

1. A nucleic acid molecule simultaneously inhibiting a Homo sapiens MET proto-oncogene (c-MET) gene and a Programmed death-ligand 1 (PD-L1) gene, wherein a nucleotide sequence inhibiting the c-MET gene and a nucleotide sequence inhibiting PD-L1 partially form a complementary bond.

2. The nucleic acid molecule of claim 1, wherein the nucleic acid molecule comprises a nucleotide sequence having 80% or more homology to a nucleotide sequence represented by SEQ ID NO: 1 or 2.

3. The nucleic acid molecule of claim 2, wherein the nucleic acid molecule including the nucleotide sequence represented by SEQ ID NO: 1 inhibits the expression of the c-MET gene by RNA interference.

4. The nucleic acid molecule of claim 2, wherein the nucleic acid molecule including the nucleotide sequence represented by SEQ ID NO: 2 inhibits the expression of the PD-L1 gene by RNA interference.

5. The nucleic acid molecule of claim 2, wherein the nucleic acid molecule is double-stranded siRNA in which small interfering RNA (siRNA) containing the nucleotide sequence represented by SEQ ID NO: 1 and siRNA containing the nucleotide sequence represented by SEQ ID NO: 2 partially form a complementary bond.

6. The nucleic acid molecule of claim 2, wherein the nucleotide sequence represented by SEQ ID NO: 1 and the nucleotide sequence represented by SEQ ID NO: 2 partially form a complementary bond to have a hairpin structure.

7. The nucleic acid molecule of claim 2, wherein the nucleic acid molecule is short hairpin RNA (shRNA) comprising the nucleotide sequence represented by SEQ ID NO: 1 and the nucleotide sequence represented by SEQ ID NO: 2.

8. The nucleic acid molecule of claim 7, wherein the shRNA comprises a nucleotide sequence having at least 80% homology to a nucleotide sequence represented by SEQ ID NO: 3 or 4.

9. A recombinant expression vector comprising and expressing the nucleic acid molecule of claim 1.

10. A virus introduced with the recombinant expression vector of claim 9.

11. The adenovirus of claim 10, wherein the virus is an adenovirus, wherein the adenovirus is adenovirus including E1A and E1B operably linked to the hTERT promoter; and a nucleotide sequence encoding SEQ ID NO: 1 and SEQ ID NO: 2 in a E3 region, wherein when the nucleotide sequence is expressed, SEQ ID NO: 1 and SEQ ID NO: 2 partially form a complementary bond to have a hairpin structure.

12. A pharmaceutical composition for preventing or treating cancer comprising the nucleic acid molecule of claim 1.

13. The pharmaceutical composition for preventing or treating cancer of claim 12, further comprising: an anti-cancer agent.

14. The pharmaceutical composition for preventing or treating cancer of claim 13, wherein the anti-cancer agent is at least one selected from the group consisting of dacomitinib, osimertinib, cetuximab, Pyrotinib, Lcotinib, panitumumab, zalutumumab, Nimotuzumab, matuzumab, gefitinib, erlotinib, Lapatinib, neratinib, vandetanib, necitumumab, afatinib, Taxol, Cisplatin, doxorubicin, paclitaxel, vincristine, topotecan, docetaxel, 5-fluorouracil (5-FU), gleevec, carboplatin, daunorubicin, valrubicin, flutamide, gemcitabine, and Etoposide.

15. (canceled)

16. A method for treating cancer, the method comprising administering the nucleic acid molecule of claim 1 to a subject in need thereof.

17. A method for treating cancer, the method comprising administering the recombinant expression vector of claim 9 to a subject in need thereof.

18. A method for treating cancer, the method comprising administering the virus of claim 10 to a subject in need thereof.

19. A pharmaceutical composition for preventing or treating cancer comprising the recombinant expression vector of claim 9.

20. A pharmaceutical composition for preventing or treating cancer comprising the virus of claim 10.
